SEQUENCE LISTING (1 sequence)

SEQ ID NO: 1 - protein, 533 aa, artificial
  mScarlet-e-NanoLuc
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGRHIDMKTVKVNITTPDGPVYDADIEMVSVRAESGDLGILPGHIPTKAPLKIGAVRLKKDGQTEMVAVSGGTVEVRPDHVTINAQAAETAEGIDKERAEAARQRAQERLNSQSDDTDIRRAELALQRALNRLDVAGKANEFMLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

(19) United States
(12) Patent Application Publication
(10) Pub. No.: US 2020/0400655 A1
(43) Pub. Date: Dec. 24, 2020

(54) RATIOMETRIC BRET MEASUREMENTS OF ATP WITH A GENETICALLY-ENCODED LUMINESCENT SENSOR

(71) Applicant: Purdue Research Foundation, West Lafayette, IN (US)

(72) Inventors: Mathew Tantama, West Lafayette, IN (US); Se-Hong Min, West Lafayette, IN (US)

(73) Assignee: Purdue Research Foundation, West Lafayette, IN (US)

(21) Appl. No.: 16/902,323

(22) Filed: Jun. 16, 2020

Related U.S. Application Data
(60) Provisional application No. 62/862,729, filed on Jun. 18, 2019.

(51) Int. Cl. G01N 33/542; G01N 33/58

(52) U.S. Cl. CPC G01N 33/542; G01N 33/582

(57) ABSTRACT

Disclosed herein is a protein-based, genetically-encoded bioluminescent sensor that can report changes in intracellular ATP in live cells. This sensor is built from the NanoLuc luciferase (Promega) and mScarlet red fluorescent protein (DOI: 10.1038/nmeth.4074) in a novel platform that provides a much more quantitative optical signal for measurements. Analytical Chemistry demonstrates its characterization and that it can be used to image ATP differences in live cells under the skin of mice. This new sensor can be very useful for research purposes in cancer biology to measure cell health and chemotherapeutic drug efficacy for example in animal models of tumors or in cell-based screening.

### GOVERNMENT SUPPORT CLAUSE

This invention was made with government support under contracts F32MH115432, R21NS092010 and UL1TR002529, awarded by the National Institute of Health. The government has certain rights in the invention.

### REFERENCE TO SEQUENCE LISTING

The present application incorporates by reference a file 68703-01_SEQ_ST25.txt including SEQ ID NO:1, provided in a computer readable form, created on Jun. 18, 2019 with a size of 5 KB, and filed with the present application. The sequence listing recorded in the file is identical to the written sequence listing provided herein.

### TECHNICAL FIELD

The present application relates to ratiometric bioluminescence resonance energy transfer (BRET) BRET measurements of ATP. Specifically, protein engineering was used to generate a new ratiometric BRET sensor of ATP that comprising an ATP-binding protein flanked by variants of the high photon flux NanoLuc luciferase as donors and a panel of red fluorescent proteins as acceptors.

### BACKGROUND

This section introduces aspects that may help facilitate a better understanding of the disclosure. Accordingly, these statements are to be read in this light and are not to be understood as admissions about what is or is not prior art.

Bioluminescence imaging has been widely adopted in biomedical research because it offers a cost effective, easily accessible method for low background and high signal-to-noise non-invasive optical measurements. For example, bioluminescent reporters have been extensively used with rodent cancer models to measure signaling, tumor volume, and immune cell infiltration in vivo. Additionally, a number of luminescent probes have been developed for metals and ions, second messengers, enzyme activity, protein-protein interactions, and reactive species, but these are predominantly used for in vitro assays, cultured cell models, or instrument development. One challenge in the development of genetically-encoded luciferase reporters is that the rapid decay of luminescence intensity over time poses a substantial problem for stable signal measurement. Furthermore, the absolute luminescence intensity varies depending on the expression levels of genetically-encoded luciferase reporters. Thus, luminescence intensity is not an ideal readout for quantitative comparisons between independent specimens such as cell assay treatment groups and animal models.

In contrast to intensity-based reporters, ratiometric sensors that employ intramolecular bioluminescence resonance energy transfer (BRET) provide an optical signal normalized for variations in luminescence flux caused by differences in sensor expression level or changing substrate concentration. However, ratiometric BRET sensors have not been widely used beyond in vitro applications, with notable exceptions such as luminescent calcium imaging in brain slices and the detection of protein-protein interactions in vivo.

There is, therefore an unmet need for novel BRET analyte sensors to be broadly used in tissue contexts with the quantitative advantages of ratiometric measurements.

### SUMMARY

A system is disclosed that comprises a construct encoding a bioluminescence resonance energy transfer (BRET) sensor for measurement of ATP levels in live cells, tissues or animals, and the construct comprises DNA sequence for mScarlet-ε-NanoLuc SEQ ID NO:1.

A method to measure ATP levels in live cells, tissues or animals is disclosed. The method comprising:
Transfecting the aforementioned DNA construct into the live cells, tissues or animals;
Incubating for about 24-48 hours for sufficient mScarlet-ε-NanoLuc protein expression;
Isolating cells expressing said mScarlet-ε-NanoLuc protein;
imaging Bioluminescence of NanoLuc and mScarlet using 450/50 and 620/15 nm emission filters respectively; and
Obtaining BRET efficiency (ATP level) by calculating the emission ratio of mScarlet/NanoLuc.

BRIEF DESCRIPTION OF SEQUENCE LISTING

| | |
|---|---|
| SEQ ID NO: 1 complete sequence of mScarlet-ϵ-NanoLuc: | |
| MVSKGEAVIK EFMRFKVHME GSMNGHEFEI EGEGEGRPYE GTQTAKLKVT KGGPLPFSWD | 60 |
| ILSPQFMYGS RAFTKHPADI PDYYKQSFPE GFKWERVMNF EDGGAVTVTQ DTSLEDGTLI | 120 |
| YKVKLRGTNF PPDGPVMQKK TMGWEASTER LYPEDGVLKG DIKMALRLKD GGRYLADFKT | 180 |
| TYKAKKPVQM PGAYNVDRKL DITSHNEDYT VVEQYERSEG RHSTGRHIDM KTVKVNITTP | 240 |
| DGPVYDADIE MVSVRAESGD LGILPGHIPT KAPLKIGAVR LKKDGQTEMV AVSGGTVEVR | 300 |
| PDHVTINAQA AETAEGIDKE RAEAARQRAQ ERLNSQSDDT DIRRAELALQ RALNRLDVAG | 360 |
| KANEFMLEDF VGDWRQTAGY NLDQVLEQGG VSSLFQNLGV SVTPIQRIVL SGENGLKIDI | 420 |
| HVIIPYEGLS GDQMGQIEKI FKVVYPVDDH HFKVILHYGT LVIDGVTPNM IDYFGRPYEG | 480 |
| IAVFDGKKIT VTGTLWNGNK IIDERLINPD GSLLFRVTIN GVTGWRLCER ILA | |

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1. Sensor designs. Diagrams of a BRET-only sensor (A) or BRET-FRET sensor (B) depict NanoLuc in blue, the B. subtilis e subunit in grey, an RFP in red, and either mNeonGreen or mTurquoise2 in green.

FIG. 2. Protein characterization of the mScarlet-epsilon-NanoLuc sensor. (A) Luminescence emission spectra and (B) the ATP-dependent mScarlet/NanoLuc BRET ratio response (K_{D}=1.1±0.1 mM, n=3) were measured on a microplate reader and data were fitted to the Hill equation. (C-D) The BRET ratio (purple) is stable over time even while the total luminescence intensity (blue) decays rapidly. The BRET ratio is (C) 0.0063±0.0001 in the presence of 0 mM ATP and (D) 0.0324±0.009 in the presence of 10 mM ATP (n=3).

FIG. 3. Live single-cell BRET microscopy. (A-B) HEK293A cells expressing the mScarlet-epsilon-NanoLuc sensor were subjected to either metabolic inhibition with 10 mM 2DG and 2.5 μM oligomycin A (Drug) or 10 mM glucose and DMSO (Veh) just prior to acquisition marked by the arrowhead. (A) BRET ratio images for typical cells undergoing each treatment at different time points. Scale bar: 10 μm. (B) The sensor BRET ratio faithfully reports the decrease in cellular ATP in inhibitor-treated cells (Drug, n=4 independent wells, 7-9 cells/well) and no change in the glucose control (Veh, n=3). Error bars represent mean±sd. (C) The BRET ratio (purple) is stable over time even as total luminescence intensity decays (blue). The BRET ratio (purple) is the same data shown in (B).

FIG. 4. Ratiometric BRET imaging of mScarlet-ε-NanoLuc purified protein using the SI Ami HT whole-animal imaging system. (A) The dynamic range is not affected by use of standard GFP (510/20 nm) and RFP (650/20 nm) emission filters in the Spectral Ami imaging system (K_{D}=1.1±0.2 mM, n=3). Data were fitted to the Hill equation. (B-C) The mScarlet/NanoLuc BRET ratio (purple) is stable over time in solutions containing 0 mM ATP (B) or 10 mM ATP (C) and 1 nM mScarlet-ε-NanoLuc protein (n=4) while the total luminescence intensity (blue) decays. (D-E) Whole-animal imaging of unshaved adult Balb/c mice. mScarlet-ε-NanoLuc pre-equilibrated with or without ATP was placed subcutaneously in the left and right hindlimb areas, respectively, and the ATP-dependent ratio difference was preserved when imaged through tissue and fur (mean±sd, n=3, p<0.001).

FIG. 5. The mScarlet-ε-NanoLuc BRET ratio reports differences in energy metabolism in live cells when imaged through tissue and fur and does not depend on sensor expression levels. The mScarlet/NanoLuc BRET ratio reports differences in ATP levels in (A) adherent HEK293A cells treated with glucose or an inhibitor cocktail of 2DG and oligomycin A (mean±sd, n=6, p<0.001) or (B) live cell suspensions treated with glucose or inhibitors (mean±sd, n=5, p<0.001). (C-F) Whole-animal imaging of unshaved adult FVB mice injected with cell suspensions (n=6). (D) Colored circles and lines connect glucose- and inhibitor-treated cells for individual mice. The black horizontal line and error bars are the population mean±sd (p=0.013). There is no correlation between BRET ratio and total luminescence intensity (expression level) in the presence of glucose (E) R²=0.01 or inhibitors (F) R²=0.12.

FIG. 6. FRET ATP-dose response curves for BRET-FRET sensors. ATP dose-response data from purified BRET-FRET sensor proteins. FRET ratio was calculated by emission ratio of acceptor fluorescence protein donor fluorescence protein from each sensor protein. Plots were fitted using a Hill equation. All BRET-FRET sensors have ˜1.0 μM Kd value which is consistent with previous studies. Red square: mNeonGreen-ε-CeNL (NC), blue circle: mScarlet-ε-CeNL (SC), yellow triangle: RRvT-ε-CeNL (RC), green reverse triangle: mScarlet-ε-GeNL (SG), and purple diamond: RRvT-ε-GeNL (RG). mean±sd, n=2-3, K_{D} is 1.0±0.4 μM (NC), 1.0±0.01 μM (SC), 1.0±0.3 μM (RC), 1.0±0.2 μM (SG), 1.0±0.1 μM (RG).

FIG. 7. BRET ratio stability over time for the GRvT-NanoLuc non-ATP binding control construct. (A) A control GRvT-NanoLuc construct lacking the E subunit did not show improved BRET despite increased spectral overlap. (B) Similar to ATP-binding sensors reported in the main text, the GRvT-NanoLuc BRET ratio was stable despite the rapid luminescence intensity decay.

FIG. 8. BRET ratio stability of mScarlet-ε-NanoLuc over time and protein concentration. (A) Different concentrations (1-100 nM) of purified mScarlet-ε-NanoLuc protein. BRET ratios from all concentrations were stable despite luminescence decay until 10 min after the addition of coelenterazine-h. The BRET ratio from 100 nM protein was noisy 10 min after the addition of the substrate because the higher enzyme concentration caused faster substrate consumption and faster luminescence decay so that background luminescence contributed to decreased signal-to-noise and increased variance in the BRET ratio when analyzing. However, overall, the BRET ratio is not directly affected by luminescence intensity. Error bars represent sd (n=4). (B) Adherent HEK293A cells seeded at different densities from 20,000 cells to 1,250 cells per well in a 96-well plate. As cells were equilibrated after the addition of coelenterazine-h, the BRET ratio increased slowly for the first minutes, but it became stable despite ˜10-fold changes in luminescence intensity. Error bars represent sd (n=4).

The attached drawings are for purposes of illustration and are not necessarily to scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

Luciferase-based reporters provide a key measurement approach in a broad range of applications, from in vitro high-throughput screening to whole animal imaging. In many assays, luminescence intensity is used to measure promoter activity, protein expression levels, or cell growth. In addition, sensors have been developed that report fluctuations in analyte concentration via dynamic changes in bioluminescence resonance energy transfer (BRET). However, BRET analyte sensors have not been broadly used in tissue contexts despite the quantitative advantages of ratiometric measurements. In principle, a ratiometric BRET readout could mitigate reproducibility problems caused by signal irregularities linked to luminescence decay and variations in reporter expression level. In this study, our objective was to demonstrate that a ratiometric BRET sensor can provide a quantitative readout across protein, cell, and whole animal tissue contexts. To do so, we used protein engineering to generate a new ratiometric BRET sensor of ATP. We screened a color palette of sensors utilizing an ATP-binding protein flanked by variants of the high photon flux NanoLuc luciferase as donors and a panel of red fluorescent proteins as acceptors. We find that the combination of NanoLuc and mScarlet exhibits the largest dynamic range, with a 5-fold change in the BRET ratio upon saturation with ATP. Using this new sensor, we show that the BRET ratio is independent of luminescence intensity decay and sensor expression level, and the BRET ratio faithfully reports differences in live cells energy metabolism whether in culture or within mouse tissue.

In principle, a ratiometric BRET readout could mitigate reproducibility problems caused by signal irregularities linked to luminescence decay and variations in reporter expression level. In this study, our objective was to demonstrate that a ratiometric BRET sensor can provide a quantitative readout across protein, cell, and whole animal tissue contexts. To do so, we used protein engineering to generate a new ratiometric BRET sensor of ATP. We screened a color palette of sensors utilizing an ATP-binding protein flanked by variants of the high photon flux NanoLuc luciferase as donors and a panel of red fluorescent proteins as acceptors. We find that the combination of NanoLuc and mScarlet exhibits the largest dynamic range, with a 5-fold change in the BRET ratio upon saturation with ATP. Using this new sensor, we show that the BRET ratio is independent of luminescence intensity decay and sensor expression level, and the BRET ratio faithfully reports differences in live cell energy metabolism whether in culture or within mouse tissue.

In this study, our objective was to demonstrate that a ratiometric BRET sensor provides an effective tool for live-cell measurements across model systems, from single-cell microscopy of cultured cells to macroscopic imaging through animal tissue. As our test case, we engineered a bioluminescent ATP sensor because of the broad importance of bioenergetic status as a fundamental metric of physiology. Furthermore, we showed that it is possible to use a red fluorescent protein (RFP) acceptor to create a BRET sensor with a large donor-acceptor emission wavelength difference that facilitates filter-based imaging in widely available animal imaging systems.

### Experimental Section

Materials. Unless otherwise noted, chemicals were purchased from Sigma, enzymes from New England Biolabs, and cell culture reagents from ThermoFisher. Coelenterazine-h was purchased from Biotium and Nano-Glo furimazine reagent from Promega.

Protein Engineering and Library Screen. Sensors were constructed by Gibson Assembly using the NEB HiFi kit, sub-cloned into the pRSETB vector for expression as His-tagged protein in BL21(DE3) E. coli, and purified by nickel-affinity chromatography. CeNL/pcDNA3 was a gift from Takeharu Nagai (Addgene plasmid #85199), pcDNA3-mNeonGreen was a gift from Richard Day (Indiana University), pCytERm-mScarlet_N1 was a gift from Dorus Gadella (Addgene plasmid #85066), and pBad-HisB-RRvT (Addgene plasmid #87364) and pBad-HisB-GRvT (Addgene plasmid #87363) were gifts from Robert Campbell. The fluorescence and luminescence ATP dose-response curves for purified protein solutions were measured on a BioTek Synergy H4 multi-mode microplate reader at ambient temperature. Luminescence was also measured using a Spectral Instruments Ami HT. Plasmids generated in this study are distributed via Addgene.

Live single-cell microscopy. The sensor genes were sub-cloned into the GW1 mammalian expression vector, and HEK293A cells were transfected by the calcium phosphate method. Cells were imaged with 30 second exposure times and 2 minute imaging interval on an Olympus IX83 microscope with a 40× 1.35 NA Apo oil objective, an Andor Xyla 4.2 sCMOS camera at 2-by-2 pixel binning, and 470/24 nm and 632/60 nm emission filters. Just prior to the addition of furimazine, well volume was exchanged twice with imaging media without glucose. After adding furimazine, a three point baseline was acquired. Subsequently, 10 mM 2-deoxyglucose (2DG) plus 2.5 μM oligomycin A was added for metabolic inhibition or 10 mM glucose plus DMSO was added for vehicle controls. Cells were imaged for ˜30 minutes until luminescence decayed to background. Image backgrounds were subtracted before calculating the ratio images of the luminescence channels using ImageJ.

Animal Imaging System. All animal procedures were performed in strict accordance with recommendations provided in the National Institutes of Health (NIH) Guide for the Care and Use of Laboratory Animals, according to protocols approved by the Purdue Animal Care and Use Committee and the Purdue University Laboratory Animal Program to minimize pain and suffering. Male and female adult Balb/c and FVB mice were sacrificed and then injected with protein solution or live cells subcutaneously in the hind limb area. NanoLuc substrate, either 20 μM coelenterazine-h or 0.5× Promega Nano-Glo furimazine solution, was mixed with the sample just prior to injection. Protein, cells, and animals were imaged at ambient temperature. Preliminary tests showed that ratiometric bioluminescent imaging could be easily carried out on both Spectral Instruments Ami HT (SI Ami HT) and Perkin Elmer IVIS Lumina II animal imaging systems with similar results, demonstrating that the sensor can be used in general across imaging systems. Therefore, in this study we used the SI Ami HT equipped with a 510/20 nm and 650/20 nm emission filters for all subsequent experiments.

Data Analysis. All measurements are shown as mean±standard deviation (sd). Unpaired, two-tailed Student's t-test was used for comparison between the means of two groups.

Supporting Methods

Plasmid Construction.

The sensor constructs were cloned into the XhoI/HindIII sites of pRSETB bacterial expression vector or the XbaI/EcoRI sites of GW1 mammalian expression vector. The Bacillus subtilis E subunit cDNA was amplified from a pRSET-ATeam1.03. NanoLuc cDNAs were amplified from Addgene plasmid #83926. Modified cDNAs containing mNeonGreen were amplified from a plasmid pcDNA3-mNeonGreen provided as a gift from Richard Day (Indiana University). CeNL cDNA was amplified from a plasmid pcDNA-CeNL (Addgene plasmid #85199) provided as a gift from Takeharu Nagai. Modified cDNAs for mScarlet were amplified from a plasmid pCytERm_mScarlet_N1 (Addgene plasmid #85066) provided as a gift from Dorus Gadella. A modified cDNAs for GRvT and RRvT were amplified from plasmids pBad-HisB-GRvT (Addgene plasmid #87363) and pBad-HisB-RRvT (Addgene plasmid #87364) provided as a gift from Robert Campbell. All sensors were constructed using standard cloning procedures. The cDNAs were amplified by PCR using Q5 polymerase (NEB), and vectors were double digested with restriction enzymes (NEB). Gibson assembly using NEB HiFi kit was used to assemble the plasmids for transformation. Plasmids generated in this study are distributed via Addgene.

Library Screen and Characterization of Purified Sensor In Vitro.

The fluorescence and luminescence of purified sensor proteins were investigated in assay buffer (50 mM MOPS-KOH, 50 mM KCl, 0.5 mM MgCl2, and 0.05% Triton X-100, pH 7.3) in a 96-well plate using a BioTek Synergy H4 multi-mode microplate reader at ambient temperature. For FRET library screening, 420/50 and 485/20 nm excitation filters and 485/20, 528/20, and 620/15 nm emission filters were used. For BRET screening, 20 μM coelenterazine-h was used as a substrate of NanoLuc and 450/50, 485/20, 528/20, and 620/15 nm emission filters were used. For characterization of mScarlet-ε-NanoLuc, 20 μM coelenterazine-h was applied to a reaction solution containing the purified sensor protein and 0-10 mM ATP in assay buffer. Luminescence spectra were scanned from 400 to 650 nm. Emissions of NanoLuc and mScarlet were measured using 450/50 and 620/15 nm emission filters, respectively, and the BRET efficiency was obtained by calculating the emission ratio of mScarlet/NanoLuc. Luminescence was also measured using a Spectral Instruments Ami HT with 510/20 and 650/20 nm emission filters (binning 2×2, FOV 25 cm, f-stop 1.2). Purified mScarlet-ε-NanoLuc protein in assay buffer with 0-10 mM ATP was imaged with a 1 sec exposure time after the addition of 20 μM coelenterazine-h. For measurements at different concentrations (1-100 nM) of mScarlet-ε-NanoLuc protein, the purified protein was mixed with 10 mM ATP or 0 mM ATP and imaged after the addition of 20 μM coelenterazine-h with a 5 sec exposure time for a total 30 min. Regions of interest were drawn over each well for all filter sets, and the average radiance was determined.

HEK293A Cell Maintenance.

HEK293A cells were cultured in Dulbecco's modified Eagle's medium containing 4.5 g/L glucose, 2 mM glutamine, and supplemented with 10% cosmic calf serum (HyClone) and were maintained at 37° C. in a 5% CO2 humidified incubator. Cells were transfected with plasmids carrying mScarlet-NanoLuc by the calcium phosphate method.

Live Single-Cell BRET Microscopy

HEK293A cells seeded onto glass-bottomed 96 well plates were allowed to adhere for two days before being transfected with sensor DNA. Cells were imaged 36-48 hours later on an Olympus IX83 microscope with an Andor Xyla 4.2 sCMOS camera, a Lumencor LED light engine, and a Prior motorized stage controlled by Andor iQ3 software. NanoLuc bioluminescence and mScarlet BRET were observed using 470/24m ET (Chroma) and 632/60m ET (Chroma) filters, respectively. An Olympus UApo N 340 40× objective (1.35 NA) was used for capture with exposure time 30 s and 2×2 binning each channel.

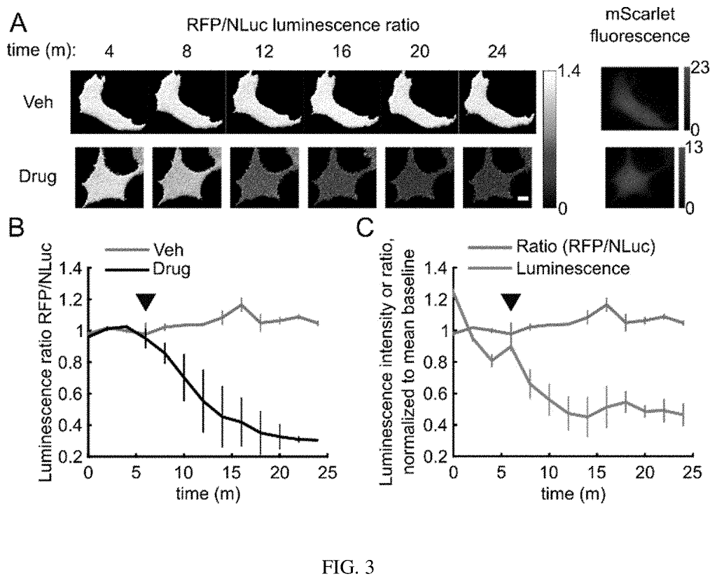

After removing the plate from the incubator, cell growth media was exchanged for imaging media (15 mM HEPES, 120 mM NaCl, 3 mM KCl, 3 mM NaHCO₃, 1.25 mM NaH2PO4, 10 mM glucose, 2 mM CaCl2, 1 mM MgCl2). Imaging regions were determined using mScarlet fluorescence and DIC. Cell media was then exchanged twice for imaging media lacking glucose. Following this, Nano-Glo substrate (Promega #N2011) was prepared according to the manufacturer's guidelines with 1× furimazine concentration and added to the well. Cells were imaged for a three point baseline (t=2 m) before having either metabolic inhibitors (10 mM 2DG+2.5 μM oligomycin A) or vehicle (10 mM glucose+DMSO) treatments mixed into the wells. Cells were imaged until luminescence decayed to background (20-30 minutes). Image backgrounds were subtracted (rolling ball radus 100 pixels) before calculating the ratio images of the luminescence channels using ImageJ. ROIs were used to calculate each cell's mean ratio from the ratio image. For FIG. 3, binary masks were generated using channel luminescence to remove pixels in ratio image corresponding to background. This is because the presence of zero value pixels in the luminescence background creates non-real pixel values in the ratio image. After normalizing each cell to its own baseline, the average time course of 6-9 cells in an independently transfected well was used as a single rep. Four reps were acquired for the drug condition and three for the vehicle.

Bioluminescence Cell Imaging.

HEK293A cells were transfected with plasmids carrying mScarlet-ε-NanoLuc by calcium phosphate method. One day after transfection, cells were passaged onto a 96-well plate at different cell densities from 1,250 to 20,000 cells per well. After an additional 24-48 hours, imaging was performed using a spectral Ami with 510/20 nm and 650/20 nm filters (5 sec exposure time, binning 2×2, FOV 25 cm, f-stop 1.2) immediately after the addition of 20 μM coelenterazine-h.

Bioluminescence Imaging of Mice.

For imaging of purified protein in mice, mScarlet-ε-NanoLuc protein pre-equilibrated with 0 mM or 10 mM ATP was mixed with 20 μM coelenterazine-h. The reaction mixture was enclosed in a custom plastic tube and placed subcutaneously in the left (0 mM ATP) and right (10 mM ATP) hindlimb areas of adult Balb/c mice carcass. The sequential imaging was immediately performed using a spectral Ami with 510/20 and 650/20 nm filters (30 sec exposure time, binning 2×2, FOV 25, f-stop 1.2). For imaging of live cells in mice, HEK293A cells expressing mScarlet-ε-NanoLuc were imaged 36-60 hours after transfection by calcium phosphate method. On the day of imaging, cells were dissociated by cell dissociation buffer (Gibco), pelleted, and resuspended in glucose-free DMEM at one million cells in 100 μL two days after transfection. The cell suspension was mixed with glucose and 10% CCS or metabolic inhibitors and incubated for 30 min at room temperature before imaged. Half of the manufacturer's suggested amount of Nano-Glo furimazine was added to the cell suspension mix and cells were imaged at a 96-well plate before they were injected into animal. The cell suspension mixture was injected subcutaneously into the fore- or hind limb areas of unshaved adult FVB mice carcass. Prior to imaging of the mice, additional half of the manufacturer's suggested amount of Nano-Glo furimazine was added. Mice were imaged immediately using a Spectral Ami at room temperature. Sequential imaging was performed using 510/20 and 650/20 nm filters (60 sec exposure time, binning 2×2, FOV 25, f-stop 1.2).

Data Analysis.

Screening data acquired on the Synergy plate reader was acquired using Gen5 and analyzed using Microsoft Excel. Live cell microscopy data was acquired using Andor iQ3 software and processed in ImageJ. Spectral Ami images were acquired from Aura imaging software (Spectral Instruments Imaging) and analyzed using ImageJ. A threshold mask was applied to eliminate background pixels. Pixel values obtained from ImageJ were converted to radiance (photons/sec/cm²/sr) by multiplying conversion factor calculated from Aura software. All measurement data were analyzed and visualized using OriginPro 2018b, MATLAB R2017a, and/or Inkscape 0.91. All measurements are shown as mean±sd. Unpaired, two-tailed Student's t-test was used for comparison between the means of two groups.

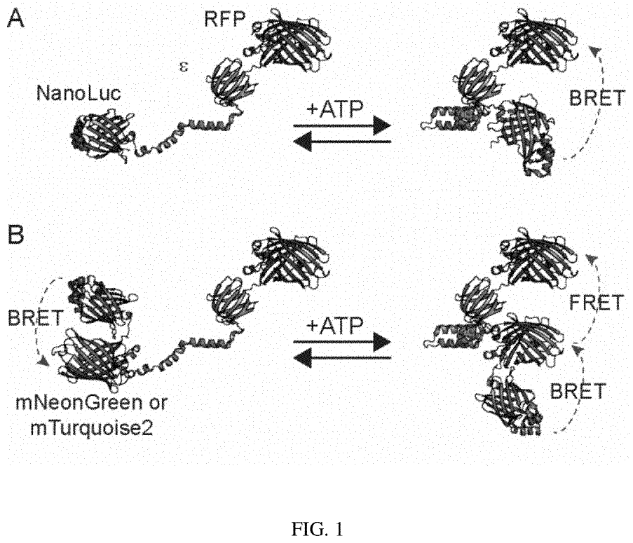

Protein Engineering and Library Screen. We first re-engineered the fluorescent Förster-type resonance energy transfer (FRET)-based ATeam1.03 ATP sensor into a color palette of BRET sensors. The ATeam1.03 sensor consists of a cyan fluorescent protein (CFP) and yellow fluorescent protein (YFP) FRET pair flanking the ATP-binding subunit of the ATP synthase from Bacillus subtilis. When ATP binds to the epsilon subunit, a large conformational change brings the fluorescent proteins into close proximity, increasing FRET. To generate our library of BRET sensors, we replaced the CFP-YFP pair with a bioluminescent donor and fluorescent protein acceptor (FIG. 1).

For the acceptor fluorescent protein, we tested tdTomato, RRvT, GRvT, and mScarlet because these RFPs provide a large spectral distance between donor and acceptor luminescence peaks, facilitating filter-based imaging. We chose tdTomato because of its potential for efficient BRET and its derivative, RRvT, because of its increased brightness. We also tested the long Stokes shift tdTomato-derivative, GRvT, given its large spectral overlap with NanoLuc. Lastly, we chose mScarlet because it is the brightest monomeric RFP to date, and it can serve as an efficient FRET acceptor to cyan-emitting donors in the same spectral range as NanoLuc emission.

We screened these acceptors in combination with three different bioluminescent donors that we can classify into two designs: BRET-only sensors and BRET-FRET dual-mode sensors (FIG. 1).

In our first design, the BRET-only sensors use NanoLuc alone as the bioluminescent donor. We tested different orientations, with NanoLuc on either the N- or C-terminus. Interestingly, we found that constructs with NanoLuc on the N-terminus and the RFP acceptor on the C-terminus of the ε subunit typically led to poor or no expression in bacteria. Thus, in our second design we only generated BRET-FRET sensors with the RFP on the N-terminus of the ε subunit.

The BRET-FRET sensors utilize one of the fluorescent protein-NanoLuc fusions, CeNL (mTurquoise2-NanoLuc) or GeNL (mNeonGreen-NanoLuc) because they have higher brightness compared to NanoLuc alone. These CeNL and GeNL-based sensors enable dual modality imaging as BRET-FRET sensors because of the FRET between the RFP and the mTurquoise2 or mNeonGreen. For comparison, we also generated the mNeonGreen-CeNL pairing because mNeonGreen and mTurquoise2 can act as a high efficiency FRET pair.

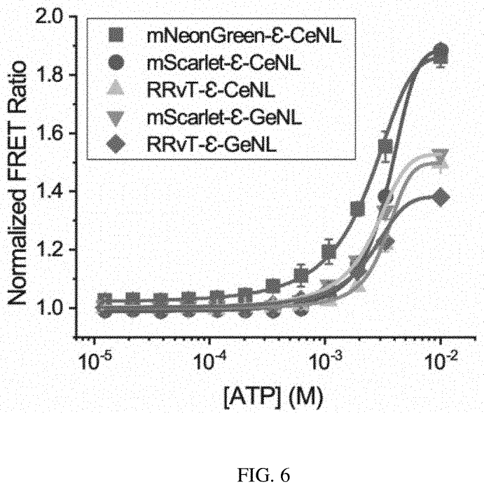

In the initial screen of these sensors, we first measured the FRET and BRET dynamic ranges after the addition of saturating ATP (Table 1). Many of the CeNL and GeNL-utilizing BRET-FRET constructs exhibited dynamic ranges with a near 100% maximal change, similar to the original ATeam1.03. Furthermore, the ATP affinity was not affected by the changes in donor and acceptor, underscoring the robustness of the ε subunit's ability to tolerate substitutions at its termini (FIG. 6). Interestingly, the magnitude of the FRET dynamic range did not correlate with or predict the BRET dynamic range (Table 1), and the lower BRET dynamic ranges were not caused by higher resting BRET. For example, the mNeonGreen-CeNL pairing resulted in one of the highest FRET dynamic ranges but the lowest BRET dynamic range. Conversely, the RRvT-GeNL pairing exhibited a higher BRET dynamic range but a lower FRET dynamic range. It may be that direct interactions between the NanoLuc and RFP in the BRET mode affect the overall dynamic range in ways that cannot be predicted by the performance of the FRET pairs.

TABLE 1
Library Screening Results.

| Donor | Acceptor | Fold-Change in Ratio | |
|---|---|---|---|
| (terminus) | (terminus) | FRET | BRET |
| NanoLuc (C) | mScarlet (N) | na^{‡} | 5.12 ± 0.18 |
| NanoLuc (C) | tdTomato (N) | na | 4.83 ± 0.20 |
| NanoLuc (C) | GRvT (N) | na | 4.56 ± 0.29 |
| NanoLuc (N) | mScarlet (C) | na | 1.07 ± 0.21 |
| NanoLuc (N) | tdTomato (C) | na | — * |
| NanoLuc (N) | GRvT (C) | na | — * |
| GeNL¹ (C) | mScarlet (N) | 1.53 ± 0.01 | 1.90 ± 0.02 |
| GeNL (C) | tdTomato (N) | — * | — * |
| GeNL (C) | RRvT (N) | 1.39 ± 0.01 | 2.04 ± 0.01 |
| CeNL² (C) | mScarlet (N) | 1.88 ± 0.01 | 1.96 ± 0.01 |
| CeNL (C) | tdTomato (N) | — * | — * |
| CeNL (C) | RRvT (N) | 1.50 ± 0.01 | 2.07 ± 0.02 |
| CeNL (C) | mNeonGreen (N) | 1.86 ± 0.06 | 1.25 ± 0.02 |

^{‡}Not applicable to BRET-only sensors.
* Poor or no expression.
GeNL is mNeonGreen-NanoLuc.
²CeNL is mTurquoise2-NanoLuc.

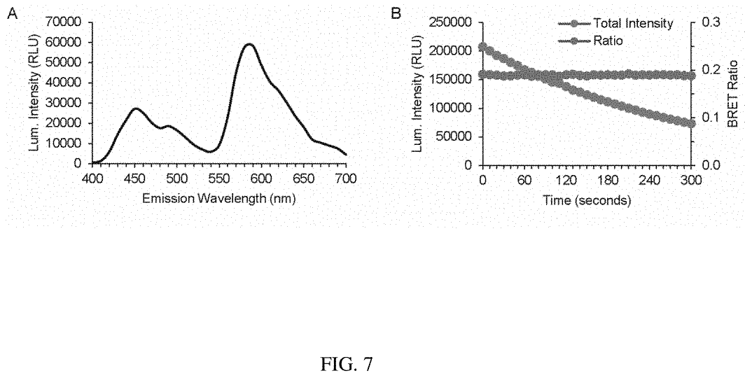

Importantly, we found that the BRET-only sensors exhibited excellent dynamic ranges of >400%, exceeding the already good performance of the BRET-FRET sensors (Table 1). These results are interesting given that the GeNL and CeNL are brighter than NanoLuc alone and both spectrally red-shift the NanoLuc emission to better overlap with the RFP absorption. It is possible that the lower BRET dynamic range in the GeNL and CeNL constructs is caused by the additional bulk of the mTurquoise2 or mNeonGreen, which might restrict the CeNL and GeNL in a conformation that is sub-optimal for BRET in the ATP-bound state. Similarly, the use of GRvT in the BRET-only constructs did not improve BRET with NanoLuc compared to tdTomato in the sensor or a control construct lacking the ε subunit (FIG. 7) despite greater overlap between NanoLuc emission and GRvT excitation spectra (Table 1).

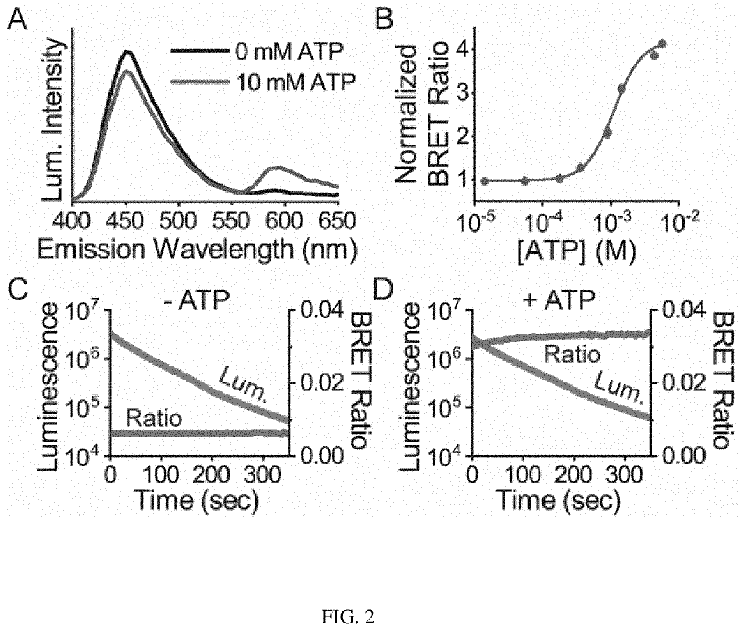

Instead, the mScarlet-NanoLuc pair worked best with a low resting BRET in the absence of ATP and a greater than 4-fold maximal change in BRET ratio upon ATP saturation (FIG. 2). Characterization of the protein in solution demonstrated that the sensor exhibited an apparent ATP affinity (K_{D}=1.1±0.1 mM, n=3) in agreement with the original ATeam1.03 at ambient temperature. Importantly, whether in the ATP-free or ATP-bound state, the BRET ratio was stable over time even during a 10-fold change in the NanoLuc intensity due to its luminescence decay (FIG. 2). The stability of the BRET ratio is a key illustration of how ratiometric measurements facilitate quantitation with reduced artifacts compared to single-channel intensity measurements that drift significantly over time.

Live Single-Cell BRET Microscopy. To test the ability of mScarlet-ε-NanoLuc to report changes in cellular ATP, we expressed this sensor in HEK293A cells and carried out luminescence imaging on a standard widefield epifluorescence microscope. Metabolic inhibition was induced by the addition of the metabolic inhibitors 2DG and oligomycin A (FIG. 3). At ambient temperature, metabolic inhibition caused a decrease in intracellular ATP levels within minutes that was reported as a −71% change in the absolute BRET ratio. This change is in good agreement with the 5-fold change from zero to saturating ATP in measurements with purified sensor (Table 1). In contrast, the BRET ratio did not decrease in the glucose vehicle control (FIG. 3).

Similar to the performance of the purified sensor protein (FIG. 2), the BRET ratio of mScarlet-ε-NanoLuc in vehicle-treated cells remains steady despite luminescence decay (FIG. 3). Furthermore, integration times of only 30 seconds were sufficient to collect ample luminescence signal with an air-cooled sCMOS camera. Thus, the use of the bright NanoLuc luciferase simplifies imaging requirements compared to other luciferases that can require minutes of integration time, back-illuminated or electron-multiplying CCD cameras, or a combination of both. Together, these results underscore the brightness and signal stability afforded by our ratiometric sensor design.

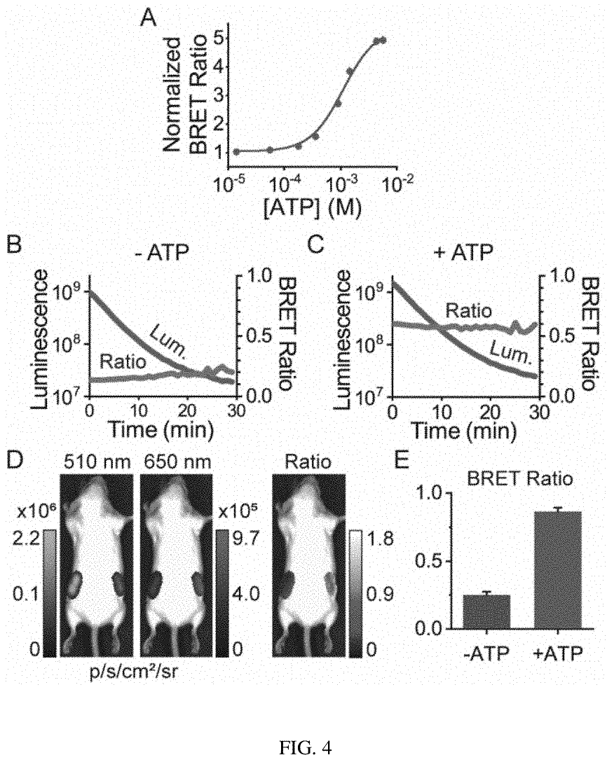

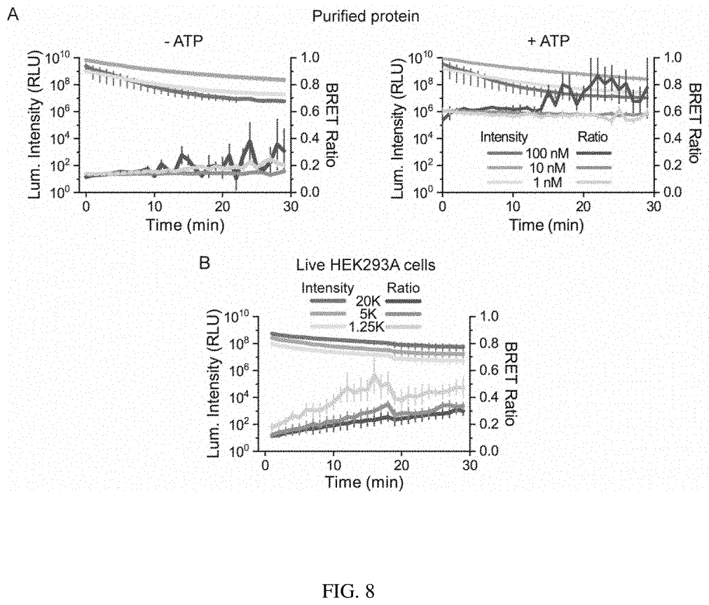

Macroscopic Imaging through Animal Tissue. We next demonstrated that the mScarlet-ε-NanoLuc sensor could serve as a ratiometric reporter of intracellular ATP when imaged through tissue. Our first objective was to characterize the spectral compatibility of the sensor with whole-animal imaging systems equipped with standard GFP and RFP emission filters that would be commonly accessible to individual labs or core facility users. In particular, the GFP filter collects emission that is off peak for the NanoLuc donor luminescence, but given the high photon flux of NanoLuc, we hypothesized that our ratio signal would not be compromised despite instrumental losses. Indeed, we found that even with the imperfect emission filter matching in the SI Ami HT system, the mScarlet-ε-NanoLuc sensor maintained a 5-fold dynamic range in its ATP dose response using protein solutions (FIG. 4). We also validated that the system could efficiently detect different levels of photon flux without causing artifacts in the BRET ratio, and in doing so we demonstrated that the BRET ratio faithfully reported differences in ATP levels in a protein concentration-independent manner (FIG. 8). Furthermore, we found the BRET ratio was stable over time despite luminescence decay, and we were able to easily measure luminescence even at 1 nM protein concentrations (FIG. 4). Thus, the mScarlet-ε-NanoLuc is bright enough that it can be effectively used for ratio imaging even without perfect wavelength-matching in the detection system.

Given these promising results, we next sought to establish whether the ATP-dependent ratio signal from mScarlet-ε-NanoLuc is preserved when imaging through tissue in a whole animal. Xenograft cancer models in which tumor cells are injected and grow as a mass below the skin are one of the most common uses of bioluminescence imaging, and therefore we carried out ratiometric imaging of subcutaneous sites in mice. We prepared purified mScarlet-ε-NanoLuc protein pre-equilibrated with or without ATP and mixed with coelenterazine-h substrate just prior to imaging. The protein was enclosed in a custom plastic tube and placed subcutaneously in the left and right hindlimb areas, respectively, of a whole mouse (FIG. 4). Even without shaving the animal, we measured a greater than 3-fold difference in the ratio, showing that the ATP-dependent signal was well-preserved when imaging through the tissue and fur (FIG. 4). Thus, we established that mScarlet-ε-NanoLuc inherently excellent for filter-based ratiometric whole-animal imaging.

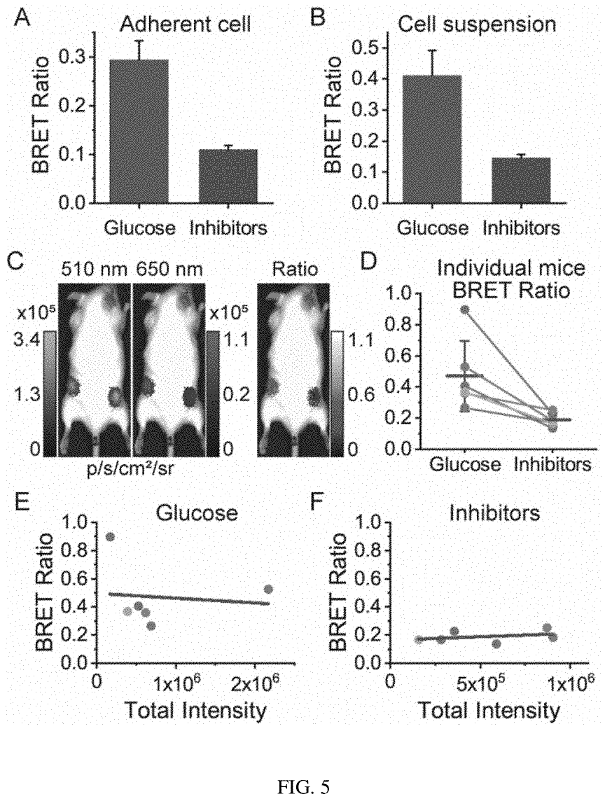

We then moved to demonstrate that mScarlet-ε-NanoLuc maintains its ability to detect metabolic differences in live cells in the context of whole animal imaging. We first validated that we could measure differences in energy metabolism using adherent cell cultures. HEK293A cells expressing mScarlet-ε-NanoLuc were seeded at different densities, from 62,500 cells/cm² down to 4,000 cell/cm² to determine the sensitivity of detection in the Spectral Ami system. As expected, when imaging live cells, we found that the addition of glucose resulted in a ˜3-fold difference in mScarlet-ε-NanoLuc ratio signal (0.29±0.04, n=6) compared to treatment with the metabolic inhibitors 2DG and oligomycin A (0.11±0.01, n=6) (FIG. 5). Thus, the sensor effectively detects metabolic differences in live cells in a dish within the animal imager.

We next carried out ratiometric imaging of live cells injected in whole animals. Live cell suspensions of sensor-expressing HEK293A cells were obtained from independent cultures for each animal. Each cell suspension was split and treated with either glucose or metabolic inhibitors in parallel. Then, similar to previous studies 10⁶ live cells were injected subcutaneously in the left and right hindlimb areas of adult FVB mice, respectively (FIG. 5). Importantly, we measured the same ratio difference between glucose and inhibitor-treated cells that was observed for cell suspensions in a dish, even through the tissue and fur of unshaved mice (FIG. 5). Of note, we used transiently transfected HEK293A cells in order to obtain a distribution of sensor expression levels across the independent trials. The total luminescence intensity provides an indicator of the total protein expression level, and critically we observed that the BRET ratio was dependent on the metabolic condition only and was independent of the level of luminescence intensity (FIG. 5). Thus, we have demonstrated that ratiometric imaging of the mScarlet-ε-NanoLuc sensor can be used to measure bioenergetic differences in live cells through tissue and fur in a manner that is independent of luminescence intensity decay and sensor expression levels.

### CONCLUSIONS

In this study, we constructed and screened a library of new luminescent BRET sensors, and we identified mScarlet-ε-NanoLuc as a sensor with a much wider spectral difference between donor and acceptor peaks compared to previous sensors. We showed that the BRET ratio is stable over time with different concentrations of protein and different numbers of cells, even when luminescence intensity decays due to substrate depletion, indicating that ratio measurements could reduce signal drift caused by variation in any of these factors in animals. Furthermore, we showed that the spectral properties of mScarlet-ε-NanoLuc facilitate its detection in commercially-available filter-based whole-animal imaging systems that are commonly accessible, and we demonstrated that it reports metabolic differences in live cells when imaged through tissue and fur of whole adult mice. Furthermore, the conversion of FRET-based sensors to BRET-based sensors can be generally considered for improved quantitation in ratiometric whole-animal imaging. This approach will become even more effective in combination with the significant progress being made in the development of new orthogonal and red-shifted of luciferase-luciferin pairs, which will allow better measurement of physiology in animal models.

Those skilled in the art will recognize that numerous modifications can be made to the specific implementations described above. The implementations should not be limited to the particular limitations described. Other implementations may be possible.

1. A construct encoding a bioluminescence resonance energy transfer (BRET) sensor for measurement of ATP levels in live cells, tissues or animals, said construct comprises DNA sequence for mScarlet-ε-NanoLuc SEQ ID NO:1.

2. A method to measure ATP levels in live cells, tissues or animals, comprising:
Transfecting the construct from claim 1 into said live cells, tissues or animals;
Incubating for about 24-48 hours for sufficient mScarlet-ε-NanoLuc protein expression;
Isolating cells expressing said mScarlet-ε-NanoLuc protein;
imaging Bioluminescence of NanoLuc and mScarlet using 450/50 and 620/15 nm emission filters respectively; and
Obtaining BRET efficiency (ATP level) by calculating the emission ratio of mScarlet/NanoLuc.
